(19) United States
(12) Patent Application Publication
(10) Pub. No.: US 2003/0096311 A1
(43) Pub. Date: May 22, 2003

(54) Screening method for compounds

(76) Inventors: John William Coleman, Liverpool (GB); Bernadette Mary Brooks, Chorlton (GB)

Correspondence Address: SHELDON & MAK, 9th Floor, 225 South Lake Avenue, Pasadena, CA, 91101, US

(21) Appl. No.: 10/273,018

(22) Filed: Oct. 16, 2002

Related U.S. Application Data
(63) Continuation-in-part of application No. PCT/GB01/01707, filed on Apr. 17, 2001.

(30) Foreign Application Priority Data
Apr. 17, 2000 (GB) GB 0009503.4

(51) Int. Cl. G01N 33/53; A61K 38/21

(52) U.S. Cl. 435/7.1; 424/85.6

(57) ABSTRACT

A method for determining whether a substance will induce an allergic response by interaction with IFN-γ or inhibit inflammation by interaction with IFN-γ comprising, first, providing the substance, then contacting the substance with IFN-γ, and the analyzing the IFN-γ contacted substance to determine if the substance has conjugated with or has interacted with the IFN-γ, where the determination that the substance has conjugated with or interacted with IFN-γ indicates that the substance will induce an allergic response by interaction with IFN-γ or inhibit inflammation by interaction with IFN-γ, and the determination that the substance has not conjugated with and has not interacted with IFN-γ indicates that the substance will not induce an allergic response by interaction with IFN-γ and will not inhibit inflammation by interaction with IFN-γ.

### CROSS-REFERENCE TO RELATED APPLICATION

This application is a continuation-in-part of PCT Application No. PCT/GB01/01707 filed Apr. 17, 2001, which claims priority from United Kingdom Application No. GB 0009503.4 filed Apr. 17, 2000, the contents of which are incorporated by reference in this disclosure in their entirety.

### BACKGROUND

The prevalence of allergic diseases such as hay fever, asthma and eczema has increased steadily during the latter half of the twentieth century to reach epidemic proportions. This trend has been associated to a large extent to the Western lifestyle. Further, it has been postulated that childhood infections may protect against an allergy by directing the immune response away from the allergic phenotype.

Allergic responses are mediated by the IgE antibody class. IgE sensitizes mast cells to release histamine and other biologically active amines and mediators in response to an allergen.

IgE mediated allergic responses are controlled by the action of cytokines secreted by T helper cells. IgE mediated allergic responses are induced by interleukin-4 (IL-4) and interleukin-13 (IL-13) which are produced by type 2 T helper (Th2) cells, and are counteracted by interferon-γ (IFN-γ) which is produced by type 1 T helper (Th1) cells. Further, IgE production by normal human lymphocytes is induced by IL-4 and suppressed by interferon-α, IFN-γ and prostaglandin E₂.

IFN-γ also promotes inflammation and cell-mediated immune defense. IL-4 also promotes antibody-mediated immunity and IgE mediated allergy. Furthermore, IFN-γ promotes further Th1 cell activation and inhibits Th2 cell activation, whereas, IL-4 promotes further Th2 cell activation and inhibits Th1 cell activation.

During the latter half of the twentieth century, when the prevalence of allergic diseases has been increasing, the use of antibiotics, and in particular β-lactam antibiotics, has increased dramatically. Many antibiotics are known to induce allergic responses. It has been suggested that antibiotics, including penicillin, reduce the release of IFN-γ by Th1 cells, and that penicillin reduces the release of IFN-γ by penicillin-specific-T cell clones. However, there is currently no method of predicting whether an antibiotic will cause an allergic reaction. Therefore, it would be useful to have a method of predicting whether an antibiotic will cause an allergic reaction.

### SUMMARY

According to one embodiment of the present invention, there is provided a method for determining whether a substance will induce an allergic response by interaction with IFN-γ. The method comprises, first, providing the substance, then contacting the substance with IFN-γ and next, analyzing the IFN-γ contacted substance to determine if the substance has conjugated with or has interacted with the IFN-γ. The determination that the substance has conjugated with or interacted with IFN-γ indicates that the substance will induce an allergic response by interaction with IFN-γ, and the determination that the substance has not conjugated with and has not interacted with IFN-γ indicates that the substance will not induce an allergic response by interaction with IFN-γ.

According to another embodiment of the present invention, there is provided a method for determining whether a substance will inhibit inflammation by interaction with IFN-γ. The method comprises, first, providing the substance, then contacting the substance with IFN-γ and next, analyzing the IFN-γ contacted substance to determine if the substance has conjugated with or has interacted with the IFN-γ. The determination that the substance has conjugated with or interacted with IFN-γ indicates that the substance will inhibit inflammation by interaction with IFN-γ, and the determination that the substance has not conjugated with and has not interacted with IFN-γ indicates that the substance will not inhibit inflammation by interaction with IFN-γ.

In one embodiment, the substance provided has an allergic profile that is unknown. In another embodiment, the substance is known to induce an allergic response but where the mechanism of the allergic response is unknown. In another embodiment, the substance is an antibiotic. In a preferred embodiment, the substance is selected from the group consisting of a carbapenam a cephalosporin, a cephamycins, a monobactam and a penicillin.

In one embodiment, contacting the substance with IFN-γ comprises mixing a quantity of the substance with IFN-γ in a buffer. In another embodiment, analyzing the IFN-γ contacted substance comprises performing one or more than one test selected from the group consisting of immunodetection, a binding study, an electrophoretic mobility study, and detecting a change in the biological activity of IFN-γ.

According to another embodiment of the present invention, there is provided a pharmaceutical substance packaged with a label or instruction indicating that it has been screened according to the method of the present invention.

According to another embodiment of the present invention, there is provided a kit for determining whether a substance will induce an allergic response by interaction with IFN-γ comprising an amount of IFN-γ, and instructions for performing a method according to the present invention. In one embodiment, the kit further comprises a container for contacting the substance with IFN-γ. In another embodiment, the kit further comprises one or more than one article used to determine whether the substance has conjugated with or has interacted with IFN-γ.

According to another embodiment of the present invention, there is provided a method for preventing a patient from having an allergic response by interaction with IFN-γ to a substance known to conjugate with or to interact with IFN-γ. The method comprises identifying a patient who is to be administered or who has been administered a substance known to conjugate with or to interact with IFN-γ, and administering to the patient an amount of exogenous IFN-γ suitable to replace an amount of IFN-γ that is or that will be conjugated with or interacted with the substance after administration of the substance.

According to another embodiment of the present invention, there is provided a method for treating a patient having an allergic response by interaction with IFN-γ to a substance known to conjugate with or to interact with IFN-γ. The method comprises identifying a patient who is having an allergic response by interaction with IFN-γ to a substance known to conjugate with or to interact with IFN-γ, and administering to the patient an amount of exogenous IFN-γ suitable to replace an amount of IFN-γ that has been conjugated with or interacted with the substance.

According to another embodiment of the present invention, there is provided a method for preventing a patient from having an inhibition of inflammation due to a substance known to conjugate with or to interact with IFN-γ. The method comprises identifying a patient who is to be administered or who has been administered a substance known to conjugate with or to interact with IFN-γ, and administering to the patient an amount of exogenous IFN-γ suitable to replace an amount of IFN-γ that is or that will be conjugated with or interacted with the substance after administration of the substance.

According to another embodiment of the present invention, there is provided a method for treating a patient who is having an inhibition of inflammation due to a substance known to conjugate with or to interact with IFN-γ. The method comprises identifying a patient who is having an inhibition of inflammation due to administration of a substance known to conjugate with or to interact with IFN-γ, and administering to the patient an amount of exogenous IFN-γ suitable to replace an amount of IFN-γ that is or that will be conjugated with or interacted with the substance after administration of the substance.

According to another embodiment of the present invention, there is provided a kit for performing a method of the present invention. The kit comprises an amount of IFN-γ, and instructions for performing a method according to the present invention. In a preferred embodiment, the kit further comprises an article used to administer the IFN-γ to the patient.

### FIGURES

These and other features, aspects and advantages of the present invention will become better understood with regard to the following description, appended claims, and accompanying figures where:

FIG. 1 is a Western blot illustrating the conjugation of BP to or interaction of BP to IFN-γ;

FIG. 2 is a graph illustrating the results of a determination of whether the effect of conjugation or interaction of the substance, BP, with IFN-γ has an effect on IFN-γ activity; and

FIG. 3 is a gel electrophoresis illustrating the other results of a determination of whether the effect of conjugation or interaction of the substance, BP, with IFN-γ has an effect on IFN-γ mediated up-regulation of macrophage chemoattractant protein -1 (MCP-1) mRNA expression in the human A549 lung epithelial cell line.

### DESCRIPTION

In one embodiment, the present invention is a method for determining whether a substance will induce an allergic response. In another embodiment, the present invention is a method for determining whether a substance will inhibit inflammation. Each method comprises contacting the substance with IFN-γ, and determining whether the substance has conjugated with or has interacted with IFN-γ, where conjugation with or interaction with IFN-γ indicates that the substance will induce an allergic response or will inhibit inflammation.

In another embodiment, the present invention is a pharmaceutical substance packaged with a label or instruction indicating that it has been screened according to a method of the present invention.

In another embodiment, the present invention is a method for preventing a patient from having an allergic response to a substance known to conjugate with or to interact with IFN-γ. In another embodiment, the present invention is a method for treating a patient who is having an allergic response to a substance known to conjugate with or to interact with IFN-γ. Each method comprising administering to the patient an amount of exogenous IFN-γ suitable to replace an amount of IFN-γ that is or that will be conjugated with or interacted with the substance after administration of the substance.

In another embodiment, the present invention is a method for preventing a patient from having an inhibition of inflammation due to a substance known to conjugate with or to interact with IFN-γ. In another embodiment, the present invention is a method for treating a patient who is having an inhibition of inflammation due to a substance known to conjugate with or to interact with IFN-γ. Each method comprises administering to the patient an amount of exogenous IFN-γ suitable to replace an amount of IFN-γ that is or that will be conjugated with or interacted with the substance after administration of the substance.

In another embodiment, the present invention is a kit for determining whether a substance will induce an allergic response. In another embodiment, the present invention is a kit for determining whether a substance will inhibit inflammation. Each kit comprises an amount of IFN-γ, and instructions for performing a method according to the present invention.

In another embodiment, the present invention is a kit for preventing or treating a patient from having or who is having an allergic response to a substance known to conjugate with or to interact with IFN-γ. In another embodiment, the present invention is a kit for preventing a patient from inhibition of inflammation or for treating a patient who is having inhibition of inflammation due to a substance known to conjugate with or to interact with IFN-γ. Each kit comprises an amount of IFN-γ, and instructions for performing a method according to the present invention.

The present invention is derived from our discovery that β-lactam benzyl penicillin conjugates to IFN-γ, thereby blocking the activity of IFN-γ and inhibiting Th2 cell activation, rather than blocking the release of IFN-γ as previously hypothesized. This activity of β-lactam benzyl penicillin appears to account for the induction of an allergic response by β-lactam benzyl penicillin. Further, we have discovered that β-lactam benzyl penicillin also conjugates to IL-4, though to a substantially lesser degree than to IFN-γ. The methods and kits of the present invention will now be discussed in more detail.

In one embodiment, the present invention is a method for determining whether a substance will induce an allergic response. In another embodiment, the present invention is a method for determining whether a substance will inhibit inflammation. Each method comprises, first, providing the substance. In a preferred embodiment, the substance will be one whose allergic profile is unknown. In another embodiment, the substance will be one that is known to induce an allergic response but where the mechanism of the allergic response is unknown.

In one embodiment, the substance is an antibiotic. In a preferred embodiment, the substance is a β-lactam antibiotic, such as an antibiotic selected from the group consisting of a carbapenam a cephalosporin, a cephamycins, a monobactam and a penicillin.

Next, the substance is contacted with IFN-γ. Contact can be made, for example, by mixing a quantity of the substance into a container containing IFN-γ or by mixing a quantity of IFN-γ into a container containing the substance. In a preferred embodiment, the substance is contacted with IFN-γ in a physiological or other appropriate aqueous buffer.

Then, the IFN-γ contacted substance is analyzed to determine if the substance has conjugated with or has interacted with the IFN-γ. Conjugation or interaction can occur chemically, structurally or functionally, or in another way as will be understood by those with skill in the art with reference to this disclosure. In one embodiment, the determination is made by immunodetection (with antibody) or by a binding study (such as with a label). In a preferred embodiment, the determination is made by detection of any structural change in the IFN-γ protein by one or more than one method selected from the group consisting of electrophoretic mobility studies, spectroscopic properties and changes in immunodetection of the cytokine or biological activity. The determination that the substance has conjugated with or interacted with IFN-γ indicates that the substance will induce an allergic response, or will inhibit inflammation, or both induce an allergic response and inhibit inflammation. The determination that the substance has not conjugated with and has not interacted with IFN-γ indicates that the substance will not induce an allergic response and will not inhibit inflammation.

In another embodiment, the present invention is a pharmaceutical substance packaged with a label or instruction indicating that it has been screened according to a method of the present invention. In a preferred embodiment, the label or instruction substance indicates that the substance can be used to treat an IFN-γ mediated inflammatory disease, such as rheumatoid arthritis.

In another embodiment, the present invention is a method for preventing a patient from having an allergic response to a substance known to conjugate with or to interact with IFN-γ. The method comprises, first identifying a patient who is to be administered or who has been administered a substance known to conjugate with or to interact with IFN-γ. Next, the patient is administered an amount of exogenous IFN-γ suitable to replace an amount of IFN-γ that is or that will be conjugated with or interacted with the substance after administration of the substance.

In another embodiment, the present invention is a method for treating a patient having an allergic response to a substance known to conjugate with or to interact with IFN-γ. The method comprises, first identifying a patient who is having an allergic response to a substance known to conjugate with or to interact with IFN-γ. Next, the patient is administered an amount of exogenous IFN-γ suitable to replace an amount of IFN-γ that has been conjugated with or interacted with the substance.

In another embodiment, the present invention is a method for preventing a patient from having an inhibition of inflammation due to a substance known to conjugate with or to interact with IFN-γ. The method comprises, first identifying a patient who is to be administered or who has been administered a substance known to conjugate with or to interact with IFN-γ. Next, the patient is administered an amount of exogenous IFN-γ suitable to replace an amount of IFN-γ that is or that will be conjugated with or interacted with the substance after administration of the substance.

In another embodiment, the present invention is a method for treating a patient who is having an inhibition of inflammation due to a substance known to conjugate with or to interact with IFN-γ. The method comprises, first identifying a patient who is having an inhibition of inflammation due to administration of a substance known to conjugate with or to interact with IFN-γ. Next, the patient is administered an amount of exogenous IFN-γ suitable to replace an amount of IFN-γ that is or that will be conjugated with or interacted with the substance after administration of the substance.

In another embodiment, the present invention is a kit for determining whether a substance will induce an allergic response. In another embodiment, the present invention is a kit for determining whether a substance will inhibit inflammation. Each kit comprises an amount of IFN-γ, and instructions for performing a method according to the present invention. In a preferred embodiment, each kit further comprises a container for contacting the substance with IFN-γ. In another preferred embodiment, each kit further comprises one or more than one article used to determine whether the substance has conjugated with or has interacted with IFN-γ.

In another embodiment, the present invention is a kit for preventing or treating a patient from having or who is having an allergic response to a substance known to conjugate with or to interact with IFN-γ. In another embodiment, the present invention is a kit for preventing a patient from inhibition of inflammation or for treating a patient who is having inhibition of inflammation due to a substance known to conjugate with or to interact with IFN-γ. Each kit comprises an amount of IFN-γ, and instructions for performing a method according to the present invention. In a preferred embodiment, each kit further comprises an article used to administer the IFN-γ to the patient.

### EXAMPLE I

### Determination of Whether a Substance will Induce an Allergic Response or Inhibit Inflammation

A determination was made in accordance with the method of the present invention whether benzyl penicillin (BP) will induce an allergic response or inhibit inflammation. The cell used for this method were prepared as follows.

Peripheral blood mononuclear cells (PBMC) were purified from the blood of healthy volunteers. The HMC-1 mast cell line was grown in Iscove's modified Dulbecco's medium plus 5% FCS (IF5) and subcultured weekly at 1:10. Peripheral blood monocytes (PBM) were purified from PBMC using anti-CD14-coated magnetic beads (MACS: Milteni Biotech, Bisley, UK), according to the manufacturer's instructions.

The generation of PBMC supernatants and RNA used for this method were prepared as follows. PBMC (10/ml) were incubated in RPMI-1640 medium containing TCH (ICN) with or without PHA (Sigma, 2.5 μg/ml). At each time point, samples were centrifuged for 5 min at 400 g, the supernatants collected and analyzed of IFN-γ by ELISA (IDS, Boldon, UK).

SDS-PAGE and Western Blotting techniques using for this method were performed as follows. Carrier-free recombinant human IFN-γ (PeproTech, London, UK) was made to 10 μg/ml and incubated overnight with or without BP (final concentrations 25, 5, 0.5 mg/ml). At the end of the incubation period, 5×loading buffer was added and 20 μl of each sample loaded onto SDS-polyacrylamide gels. The gels were then analyzed by Western blotting, using a rabbit polyclonal anti-BP antibody. A comparative study was conducted using IL-4 in place of IFN-γ.

TABLE 1
Natural and Recombinant IFN-γ in Cell-free Experiments

| | IFN-γ (pg/ml) | IFN-γ (pg/ml) detected with |
|---|---|---|
| Sample | detected without BP | BP |
| IFN-γ 200 pg/ml | 192 | 13 |
| IFN-γ 1000 pg/ml | 1015 | 70 |
| PBMC supernatant 1 | 152 | 0 |
| PBMC supernatant 2 | 389 | 0 |
| PBMC supernatant 3 | >400 | 92 |

Purified recombinant IFN-γ or cell-free supernatants from stimulated PBMC were incubated overnight with BP (500 μg/ml), then assayed by ELISA, Supernatant 1, PBMC stimulated for 20 hours with IL-2; supernatant 2, PBMC stimulated for 43 hours with IL-2; supernatant 3, PBMC stimulated for 43 hours with PHA. As can be seen, BP reduces the amount of IFN-γ detected by immunoassay in recombinant purified preparations and harvested cell supernatants stimulated with IL-2 (10 μg/ml) or PHA (2.5 μg/ml)

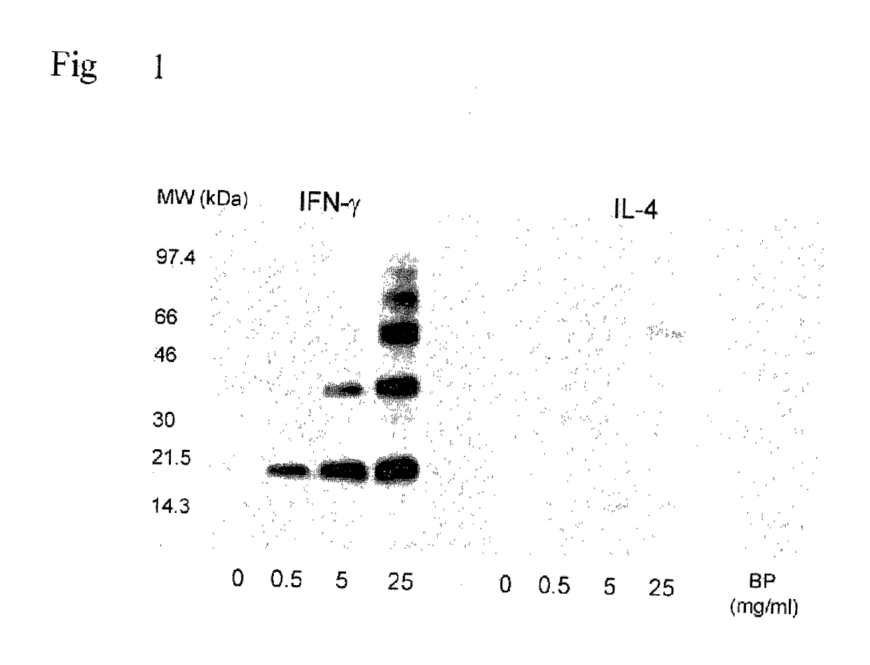

Referring now to FIG. 1, there is shown a Western blot illustrating the conjugation of BP to or interaction of BP to IFN-γ. FIG. 1 illustrates a method by which conjugation or interaction of a substance with a cytokine, namely IFN-γ, can be determined.

Carrier-free recombinant human IFN-γ (PeproTech, London, UK) were made to 10 μg/ml and incubated overnight with or without Benzyl Penicillin (BP) (final concentrations: 25, 5, 0.5 mg/ml). At the end of the incubation period, 5×loading buffer was added and 20 μl of each sample loaded onto SDS-polyacrylamide gels. For all samples, aliquots were then analyzed by Western blotting, using a rabbit polyclonal anti-BP antibody. A comparative study was conducted using IL-4 in place of IFN-γ.

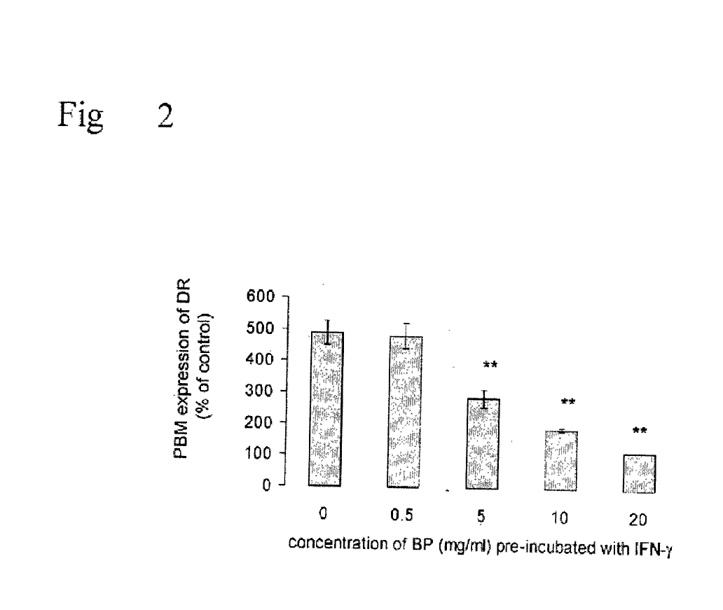

Referring now to FIG. 2, there is shown a graph illustrating the results of a determination of whether the effect of conjugation or interaction of the substance, BP, with IFN-γ has an effect on IFN-γ activity. In summary, IFN-γ was tested for induction of MHC class II DR on human peripheral blood monocytes (PBM) as follows.

IFN-γ (1 μg/ml) was incubated overnight with various concentrations of BP. HMC-1 cells were pulsed with or without IFN-γ (10 ng/ml), BP, or BP-treated IFN-γ, for 30 min and washed 3 times in PBS. The cells were resuspended in IFS and co-cultured (2×10⁵) overnight with PBM (2×10⁵) in a total volume of 200 μl in polypropylene tubes (Falcon). Six tubes were set up for each HMC-1 treatment. At the end of culture, antibodies were added as follows: 1, none; 2, anti-DR (Caltag, supplied by TCS, Buckingham, UK); 3, anti-CD45 (Caltag) plus isotype control for anti-DR; 4, anti-DR and anti-CD45 (in triplicate. Samples 1, 2 and 3 were used to set voltage gains and compensation levels. CD45 positive cells (PBM) were gated in sample 4 and the median DR fluorescence intensity measured. Results are presented as the mean±standard deviation of median fluorescence, for triplicate determinations, measured as increase in DR over control (HMC-1 pulsed without IFN-γ). As shown in FIG. 2, pre-incubation of IFN-γ with substance, BP, reduced its bioactivity in a concentration dependent manner.

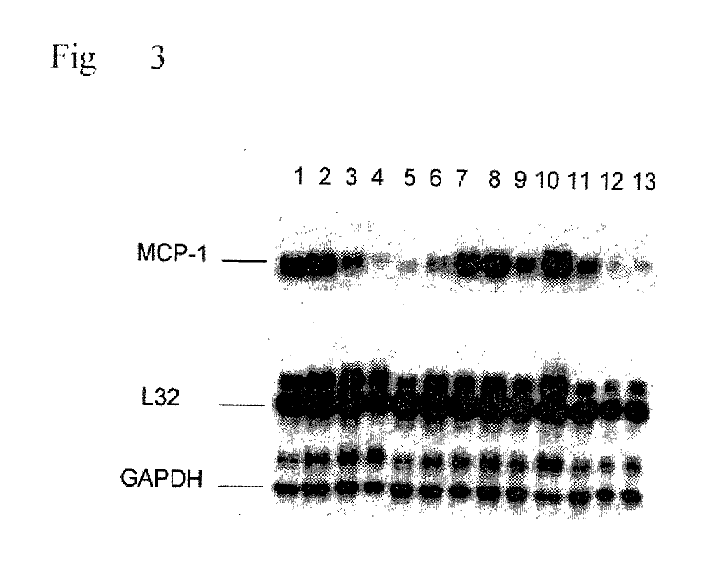

Referring now to FIG. 3, there is shown a gel electrophoresis illustrating the other results of a determination of whether the effect of conjugation or interaction of the substance, BP, with IFN-γ has an effect on IFN-γ mediated up-regulation of macrophage chemoattractant protein -1 (MCP-1) mRNA expression in the human A549 lung epithelial cell line.

IFN-γ (1 μg/ml) was incubated overnight with BP (20 or 10 mg/ml). Confluent A549 cells were incubated with IFN-γ, BP-treated IFN-γ (both diluted to final concentrations of 20, 2 and 0.2 ng/ml), BP or nothing. After two hours, RNA was extracted from the cells with TRIzol and analyzed by RNase protection.

RNase protection assays were carried out as follows. T7 RNA polymerase was used to synthesis P radiolabeled antisense RNA probes, using cDNA templates HCK 3, according to the manufacturer's instructions (Pharmingen, Cambridge, UK). Probe solutions were hybridized to 10-20 μg of total RNA at 56° C. overnight followed by digestion with RNAse A and T1 (Pharmingen). Samples were digested with proteinase K, phenol extracted and precipitated in ethanol. Each sample was loaded on to a 6% polyacrylamide urea gel, run at 38 mA in 0.5×TBE (tris-borate EDTA buffer) alongside the undigested probe as size marker. Gels were exposed overnight to autoradiography film (KODAK XAR-5) at −70° C. with intensifying screens. Densitometric analysis was carried out using the “Image” program (National Institutes of Health, USA).

As can be seen, fresh IFN-γ (lanes 7-9) and IFN-γ that had been incubated alone overnight at 37° C. (as control, lanes 1-3), induced a concentration dependent increase of MCP-1 mRNA. Pre-incubation of IFN-γ overnight with BP inhibited induction of MCP-1 mRNA (lanes 4-6). BP mixed with IFN-γ immediately before addition to the responder cells had a less marked inhibiting effect on the biological activity of the cytokine (lanes 10-12). Lane 13 shows unstimulated cells.

Although the present invention has been discussed in considerable detail with reference to certain preferred embodiments, other embodiments are possible. Therefore, the scope of the appended claims should not be limited to the description of preferred embodiments contained in this disclosure. All references cited herein are incorporated by reference to their entirety.

What is claimed is:

1. A method for determining whether a substance will induce an allergic response by interaction with IFN-γ comprising:
a) providing the substance;
b) contacting the substance with IFN-γ; and
c) analyzing the IFN-γ contacted substance to determine if the substance has conjugated with or has interacted with the IFN-γ;
where the determination that the substance has conjugated with or interacted with IFN-γ indicates that the substance will induce an allergic response by interaction with IFN-γ, and the determination that the substance has not conjugated with and has not interacted with IFN-γ indicates that the substance will not induce an allergic response by interaction with IFN-γ.

2. The method of claim 1, where the substance provided has an allergic profile that is unknown.

3. The method of claim 1, where the substance is known to induce an allergic response but where the mechanism of the allergic response is unknown.

4. The method of claim 1, where the substance is an antibiotic.

5. The method of claim 4, where the substance is selected from the group consisting of a carbapenam a cephalosporin, a cephamycins, a monobactam and a penicillin.

6. The method of claim 1, where contacting the substance with IFN-γ comprises mixing a quantity of the substance with IFN-γ in a buffer.

7. The method of claim 1, where analyzing the IFN-γ contacted substance comprises performing one or more than one test selected from the group consisting of immunodetection, a binding study, an electrophoretic mobility study, and detecting a change in the biological activity of IFN-γ.

8. A pharmaceutical substance packaged with a label or instruction indicating that it has been screened according to the method of claim 1.

9. A kit for determining whether a substance will induce an allergic response by interaction with IFN-γ comprising an amount of IFN-γ, and instructions for performing a method according to claim 1.

10. The kit of claim 9, further comprising a container for contacting the substance with IFN-γ.

11. The kit of claim 9, further comprising one or more than one article used to determine whether the substance has conjugated with or has interacted with IFN-γ.

12. A method for determining whether a substance will inhibit inflammation by interaction with IFN-γ comprising:
a) providing the substance;
b) contacting the substance with IFN-γ; and
c) analyzing the IFN-γ contacted substance to determine if the substance has conjugated with or has interacted with the IFN-γ;
where the determination that the substance has conjugated with or interacted with IFN-γ indicates that the substance will inhibit inflammation by interaction with IFN-γ, and the determination that the substance has not conjugated with and has not interacted with IFN-γ indicates that the substance will not inhibit inflammation by interaction with IFN-γ.

13. The method of claim 12, where the substance is an antibiotic.

14. The method of claim 13, where the substance is selected from the group consisting of a carbapenam a cephalosporin, a cephamycins, a monobactam and a penicillin.

15. The method of claim 12, where contacting the substance with IFN-γ comprises mixing a quantity of the substance with IFN-γ in a buffer.

16. The method of claim 12, where analyzing the IFN-γ contacted substance comprises performing one or more than one test selected from the group consisting of immunodetection, a binding study, an electrophoretic mobility study, and detecting a change in the biological activity of IFN-γ.

17. A pharmaceutical substance packaged with a label or instruction indicating that it has been screened according to the method of claim 12.

18. A kit for determining whether a substance will inhibit inflammation by interaction with IFN-γ comprising an amount of IFN-γ, and instructions for performing a method according to claim 12.

19. The kit of claim 18, further comprising a container for contacting the substance with IFN-γ.

20. The kit of claim 18, further comprising one or more than one article used to determine whether the substance has conjugated with or has interacted with IFN-γ.

21. A method for preventing a patient from having an allergic response by interaction with IFN-γ to a substance known to conjugate with or to interact with IFN-γ comprising:
a) identifying a patient who is to be administered or who has been administered a substance known to conjugate with or to interact with IFN-γ; and
b) administering to the patient an amount of exogenous IFN-γ suitable to replace an amount of IFN-γ that is or that will be conjugated with or interacted with the substance after administration of the substance.

22. A kit for preventing a patient from having or who is having an allergic response by interaction with IFN-γ to a substance known to conjugate with or to interact with IFN-γ comprising an amount of IFN-γ, and instructions for performing a method according to claim 21.

23. The kit of claim 22, further comprising an article used to administer the IFN-γ to the patient.

24. A method for treating a patient having an allergic response by interaction with IFN-γ to a substance known to conjugate with or to interact with IFN-γ comprising:
a) identifying a patient who is having an allergic response by interaction with IFN-γ to a substance known to conjugate with or to interact with IFN-γ; and
b) administering to the patient an amount of exogenous IFN-γ suitable to replace an amount of IFN-γ that has been conjugated with or interacted with the substance.

25. A kit for preventing a patient from having or who is having an allergic response by interaction with IFN-γ to a substance known to conjugate with or to interact with IFN-γ comprising an amount of IFN-γ, and instructions for performing a method according to claim 24.

26. The kit of claim 25, further comprising an article used to administer the IFN-γ to the patient.

27. A method for preventing a patient from having an inhibition of inflammation due to a substance known to conjugate with or to interact with IFN-γ comprising:
a) identifying a patient who is to be administered or who has been administered a substance known to conjugate with or to interact with IFN-γ; and
b) administering to the patient an amount of exogenous IFN-γ suitable to replace an amount of IFN-γ that is or that will be conjugated with or interacted with the substance after administration of the substance.

28. A kit for preventing a patient from inhibition of inflammation due to a substance known to conjugate with or to interact with IFN-γ comprising an amount of IFN-γ, and instructions for performing a method according to claim 27.

29. The kit of claim 28, further comprising an article used to administer the IFN-γ to the patient.

30. A method for treating a patient who is having an inhibition of inflammation due to a substance known to conjugate with or to interact with IFN-γ comprising:
a) identifying a patient who is having an inhibition of inflammation due to administration of a substance known to conjugate with or to interact with IFN-γ; and
b) administering to the patient is administered an amount of exogenous IFN-γ suitable to replace an amount of IFN-γ that is or that will be conjugated with or interacted with the substance after administration of the substance.

31. A kit for preventing or treating a patient from inhibition of inflammation or for treating a patient who is having inhibition of inflammation due to a substance known to conjugate with or to interact with IFN-γ comprising an amount of IFN-γ, and instructions for performing a method according to claim 30.

32. The kit of claim 31, further comprising an article used to administer the IFN-γ to the patient.
